(19) United States
(12) Patent Application Publication
(10) Pub. No.: US 2021/0330283 A1
(43) Pub. Date: Oct. 28, 2021

(54) ULTRASOUND IMAGE GENERATION APPARATUS, ULTRASOUND IMAGE GENERATION PROGRAM, AND ULTRASONIC DIAGNOSIS APPARATUS

(71) Applicant: Hitachi, Ltd., Tokyo (JP)

(72) Inventor: Suguru ISHIGURO, Tokyo (JP)

(21) Appl. No.: 17/190,180

(22) Filed: Mar. 2, 2021

(30) Foreign Application Priority Data
Apr. 24, 2020 (JP) 2020-077099

(51) Int. Cl. A61B 8/06; A61B 8/13; A61B 8/08; A61B 8/00

(52) U.S. Cl. CPC A61B 8/06; A61B 8/13; A61B 8/5223; A61B 8/461; A61B 8/5207

(57) ABSTRACT

In a buffer filling calculation, calculation is performed concerning an analytical model which involves performing, during a period from a first analysis time point until a second analysis time point, an operation of filling a buffer container at a constant flow rate with liquid from a liquid source, emptying the buffer container at every instance the buffer container becomes full, and continuing with the filling of the buffer container at the constant flow rate with the liquid from the liquid source. In the buffer filling calculation, a particle is generated at a flow inlet every time the buffer container becomes full, and a position of each particle at the second analysis time point is determined, which is a position at which the particle appears when the particle is moved along an inflow direction indicated by a blood flow velocity vector until the second analysis time point is reached.

### CROSS REFERENCE TO RELATED APPLICATION

This application claims priority to Japanese Patent Application No. 2020-077099 filed on Apr. 24, 2020, which is incorporated herein by reference in its entirety including the specification, claims, drawings, and abstract.

### TECHNICAL FIELD

The present disclosure relates to an ultrasound image generation apparatus, an ultrasound image generation program, and an ultrasonic diagnosis apparatus, and more particularly to a technique of generating an image concerning blood flow.

### BACKGROUND

Ultrasonic diagnosis apparatuses that measure a velocity of blood flow in an examinee's body are in wide use. Such an ultrasonic diagnosis apparatus carries out VFM (vector flow mapping), which is a process in which blood flow velocity vectors indicated by arrows or the like are displayed overlapping a tomographic image, and also performs diagnosis of circulatory system organs such as blood vessels and the heart. In an ultrasonic diagnosis apparatus that carries out VFM, blood flow velocity vectors are measured using Doppler ultrasonography.

Patent Documents 1 and 2 listed below disclose techniques in which blood flow velocity vectors obtained by VFM are represented by particles depicted on an image. According to these techniques, an interpolation frame is generated, which interpolates between an image frame generated at a preceding time point and an image frame generated at a succeeding time point. In respective images shown in image frames or interpolation frames, particles that are moved in the images along with an elapse of time are depicted. Patent Document 3 provides a description regarding a basic technique of measuring blood flow velocity.

### CITATION LIST

### Patent Literature

Patent Document 1: JP 2016-214438 A

Patent Document 2: JP 2016-202621 A

Patent Document 3: JP 2015-198777 A

According to the techniques described in Patent Documents 1 and 2, blood flow velocity vectors at respective points within the heart are expressed on an image. However, these techniques are disadvantageous, in that an image obtained by these techniques does not depict a volume of blood flowing per predetermined unit of time in a region which is not a point but rather has an extent of area; i.e., a blood flow rate in a region having an extent of area.

### SUMMARY

An object of the present disclosure is to provide, concerning a region which is a target of blood flow velocity vector analysis, an appropriate indication of a blood flow rate in a region having an extent of area.

According to an aspect of the present disclosure, an ultrasound image generation apparatus comprises a processor that executes: a process of obtaining a blood flow velocity vector at a flow inlet designated in an analysis target region; a process of calculating, based on the blood flow velocity vector, a blood inflow rate indicating an amount of blood that flows in via the flow inlet during a period from a first analysis time point until a second analysis time point; and a process of performing a buffer filling calculation based on the blood inflow rate and the blood flow velocity vector and thereby generating an ultrasound image for the second analysis time point. The buffer filling calculation is a calculation of determining a number and positions of particles to be shown in the ultrasound image based on the blood inflow rate, a pre-specified buffer capacity, and the blood flow velocity vector, and generating the ultrasound image in which the particles are shown at those positions of the particles. The buffer capacity is a value which, when a plurality of particles are to be shown in the ultrasound image, defines a distance between the particles based on the blood inflow rate.

The present disclosure enables provision, concerning a region which is a target of blood flow velocity vector analysis, of an appropriate indication of a blood flow rate in a region having an extent of area.

### BRIEF DESCRIPTION OF DRAWINGS

Embodiment(s) of the present disclosure will be described based on the following figures, wherein:

FIG. 1 is a diagram showing a configuration of an ultrasonic diagnosis apparatus;

FIG. 2 is a diagram showing an image displayed on a display unit for setting conditions for VFM;

FIG. 3 is a diagram conceptually illustrating a process of virtually designating flow inlets with respect to a blood flow opening;

FIG. 4 is a diagram showing an analytical model for buffer filling calculation;

FIG. 5 is a diagram schematically showing an ultrasound image in which particles are depicted on a tomographic image; and

FIG. 6 is a diagram schematically showing an ultrasound image in which particles are depicted on a tomographic image.

### DESCRIPTION OF EMBODIMENTS

Embodiments of the present disclosure are described below by reference to the drawings. Identical elements shown in a plurality of drawings are labeled with the same reference signs, and repeated descriptions thereof are simplified.

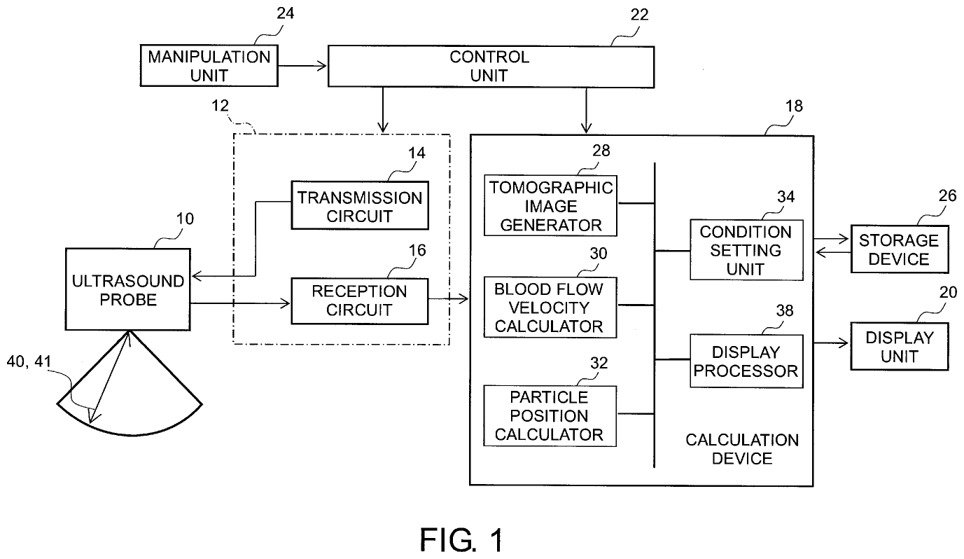

FIG. 1 shows a configuration of an ultrasonic diagnosis apparatus according to an embodiment of the present disclosure. The ultrasonic diagnosis apparatus comprises an ultrasound probe 10, a transmission/reception circuit 12, a calculation device 18, a display unit 20, a control unit 22, a manipulation unit 24, and a storage device 26. The manipulation unit 24 includes a keyboard, a mouse, a rotary knob, a lever, and the like, and serves to output, to the control unit 22, manipulation information based on manipulations performed by a user. The control unit 22 carries out overall control of the ultrasonic diagnosis apparatus based on the manipulation information. The display unit 20 may be a liquid crystal display, an organic EL display, or the like. Further, the display unit 20 may constitute a touchscreen together with the manipulation unit 24.

As the storage device 26 serving as a storage medium, a storage device such as a hard disk, a USB memory, or an SD card is used. The storage device 26 may be a storage located on a communication line such as the Internet.

The calculation device 18 comprises a tomographic image generator 28, a blood flow velocity calculator 30, a particle position calculator 32, a condition setting unit 34, and a display processor 38. The calculation device 18 may be a processor that configures these constituent elements (i.e., the tomographic image generator 28, the blood flow velocity calculator 30, the particle position calculator 32, the condition setting unit 34, and the display processor 38) in its interior by executing programs stored in an external storage medium, the storage device 26, and the like. Information to be used for calculations by the constituent elements, information that should be temporarily stored during a calculation process, information resulting from a calculation, and the like may be stored in the storage device 26.

A single constituent element of the calculation device 18 may be constituted by a plurality of processors that execute distributed processing. Further, a part or all of the plurality of constituent elements of the calculation device 18 may be constituted by an external computer. The external computer may be directly connected to the calculation device 18, or may alternatively be connected to a communication line such as the Internet. A single constituent element of the calculation device 18 may be constituted by a plurality of external computers that execute distributed processing. Further, a part or all of the plurality of constituent elements of the calculation device 18 may be individually constituted by hardware electronic circuits.

The ultrasonic diagnosis apparatus is configured to operate in the B mode for obtaining a tomographic image of an examinee's body. In the B mode, the transmission/reception circuit 12, the ultrasound probe 10, the calculation device 18, and the display unit 20 are operated as described below according to control performed by the control unit 22.

The transmission/reception circuit 12 comprises a transmission circuit 14 and a reception circuit 16. The ultrasound probe 10 comprises a plurality of vibrators. The transmission circuit 14 outputs a transmission signal to each vibrator. Each vibrator converts the transmission signal into an ultrasound wave, and transmits the ultrasound wave to an examinee's body. The transmission circuit 14 adjusts delay times of the transmission signals output to the respective vibrators in such a manner that the ultrasound waves emitted from the vibrators intensify each other in a specific direction. The transmission circuit 14 thereby forms a transmission ultrasound beam in that specific direction with the ultrasound waves, and causes the transmission ultrasound beam to scan over the examinee's body.

Each of the plurality of vibrators receives an ultrasound wave reflected from the examinee's body, converts the received ultrasound wave into an electric signal, and outputs the electric signal to the reception circuit 16. The reception circuit 16 phases and adds the electric signals output from the respective vibrators in such a manner that the electric signals based on the ultrasound waves received from the transmission ultrasound beam direction intensify each other. The reception circuit 16 thereby generates a reception signal, and outputs the reception signal to the calculation device 18. In this way, a reception ultrasound beam is formed at the ultrasound probe 10, and the reception signal generated in accordance with the reception ultrasound beam is output from the transmission/reception circuit 12 to the calculation device 18 as a reception signal that generates a tomographic image. In the following description, the term “ultrasound beam” is used as a generic term for referring to the transmission ultrasound beam and the reception ultrasound beam.

The tomographic image generator 28 configured within the calculation device 18 generates a tomographic image frame based on reception signals obtained concerning respective ultrasound beam directions according to a scan direction, and outputs the tomographic image frame to the display processor 38. The display processor 38 causes a tomographic image based on the tomographic image frame to be displayed on the display unit 20. Further, the tomographic image generator 28 stores the tomographic image frame in the storage device 26.

In the B mode, according to control performed by the control unit 22, the transmission/reception circuit 12, the ultrasound probe 10, and the calculation device 18 repeatedly carry out the scanning of an ultrasound beam 40 over the examinee's body. The tomographic image generator 28 sequentially generates tomographic image frames at a predetermined frame rate with an elapse of time, and stores those tomographic image frames in the storage device 26.

In addition to the B mode, the ultrasonic diagnosis apparatus is configured to operate in the Doppler measurement mode for obtaining blood flow velocity vectors. In the Doppler measurement mode, the transmission/reception circuit 12, the ultrasound probe 10, and the calculation device 18 are operated as described below according to control performed by the control unit 22. Here, the B mode operation and the Doppler measurement mode operation may be executed in a time-divided manner by performing, by time division, ultrasound transmission/reception according to the B mode operation and ultrasound transmission/reception according to the Doppler measurement mode operation.

The control unit 22 controls the transmission/reception circuit 12 so as to scan a transmission ultrasound beam formed by the ultrasound probe 10 and thereby transmit ultrasound waves for the Doppler measurement mode in the respective transmission ultrasound beam directions. An analysis target region to be scanned by the transmission ultrasound beam for the Doppler measurement mode may be a region included in the region scanned by the ultrasound beam 40 in the B mode. Each of the plurality of vibrators receives an ultrasound wave reflected from the examinee's body, converts the ultrasound wave into an electric signal, and outputs the electric signal to the reception circuit 16.

According to control performed by the control unit 22, the reception circuit 16 phases and adds the electric signals output from the respective ultrasound vibrators of the ultrasound probe 10 to thereby generate a reception signal for the Doppler measurement mode, and outputs the reception signal to the calculation device 18. In this way, a reception ultrasound beam is formed at the ultrasound probe 10, and the reception signal generated in accordance with the reception ultrasound beam is output from the transmission/reception circuit 12 to the calculation device 18 as a reception signal for the Doppler measurement mode.

The blood flow velocity calculator 30 configured within the calculation device 18 analyzes a Doppler shift frequency of the reception signal obtained concerning each ultrasound beam direction according to the scan direction, and determines an ultrasound beam direction component of the blood flow velocity at each position in each ultrasound beam 41 scanned in the analysis target region. By using, for example, a calculation described in Patent Document 3, the blood flow velocity calculator 30 obtains, regarding each position in each ultrasound beam 41, an orthogonal component which is orthogonal to the ultrasound beam direction component, based on the ultrasound beam direction component of the blood flow velocity at each position in each ultrasound beam 41. In Patent Document 3, the ultrasound beam direction component and the orthogonal component are referred to as “Doppler measurement component” and “cross-path direction component”, respectively.

The calculation described in Patent Document 3 is a calculation that determines, based on a differential equation that obeys the law of conservation of mass, an orthogonal component associated with an ultrasound beam direction component. Here, the law of conservation of mass is a law stating that, with respect to a given closed space, blood neither only flows in nor only flows out, but rather, blood flows out from the closed space in the same volume as the volume of blood that flowed into the closed space.

By means of the processing as described above, the blood flow velocity calculator 30 determines, for each position in the analysis target region, a blood flow velocity vector incorporating the ultrasound beam direction component and the orthogonal component. The blood flow velocity calculator 30 may subject the blood flow velocity vector to coordinate transformation processing. For example, the blood flow velocity calculator 30 may transform the blood flow velocity vector incorporating the ultrasound beam direction component and the orthogonal component into a blood flow velocity vector expressed in an orthogonal coordinate system. The blood flow velocity calculator 30 stores, in the storage device 26, the blood flow velocity vectors obtained for the respective positions in the analysis target region in the form of a blood flow velocity data set, which is further described below.

In the Doppler measurement mode, the transmission/reception circuit 12, the ultrasound probe 10, and the calculation device 18 repeatedly carry out the scanning of the ultrasound beam 41 over the examinee's body according to control performed by the control unit 22. The blood flow velocity calculator 30 sequentially generates, at a predetermined frame rate with an elapse of time, blood flow velocity data sets each representing blood flow velocity vectors obtained regarding the analysis target region, and stores those blood flow velocity data sets in the storage device 26.

As described above, the ultrasound probe 10, the transmission/reception circuit 12, and the tomographic image generator 28 constitute a tomographic image generation device that generates tomographic images based on ultrasound transmissions/receptions. Further, the ultrasound probe 10, the transmission/reception circuit 12, and the blood flow velocity calculator 30 constitute a blood flow velocity calculation device that calculates blood flow velocity vectors based on ultrasound transmissions/receptions.

The ultrasonic diagnosis apparatus operates as an ultrasound image generation device that generates ultrasound images. That is, the ultrasonic diagnosis apparatus executes VFM based on the tomographic image frames and the blood flow velocity data sets stored in the storage device 26. VFM is a process of generating, based on the tomographic image frames and the blood flow velocity data sets, data that indicate an ultrasound image in which graphics representing blood flow rates are shown overlapping a tomographic image, and causing the ultrasound image to be displayed on the display unit 20.

A blood flow rate represents a volume of blood that flows in a predetermined region per predetermined unit of time. As described further below, a blood flow rate is shown by positions and number of particles depicted on a tomographic image. Here, the term “particle” as used in the present specification signifies a graphic that expresses a flow rate. The “particle” may be a mark having a circular, polygonal, or other shape, or may be depicted as a figure such as an arrow.

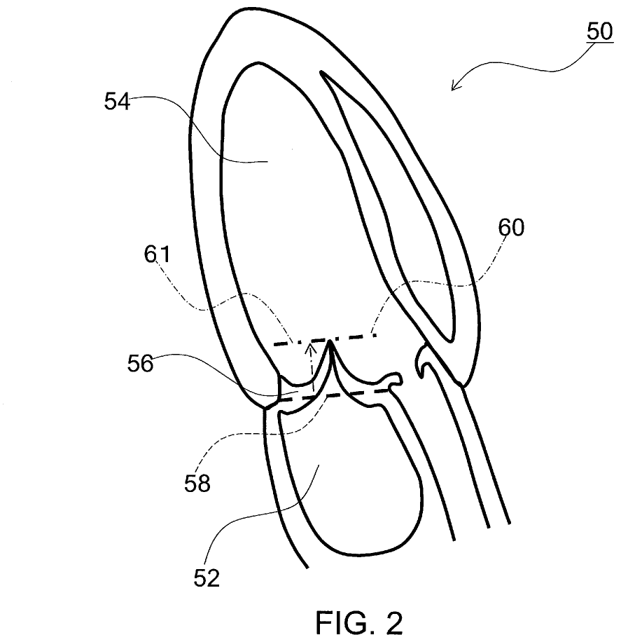

FIG. 2 shows an image that the display processor 38 causes to display on the display unit 20 for setting conditions for VFM. In FIG. 2, the left atrium 52, the left ventricle 54, and the mitral valve 56 of a heart 50 are shown. The control unit 22 controls the condition setting unit 34 in accordance with manipulations made on the manipulation unit 24, and the condition setting unit 34 sets a reference line 58 in accordance with the control performed by the control unit 22. FIG. 2 shows the reference line 58 set at the position of the mitral valve 56 so as to mark off the boundary between the left atrium 52 and the left ventricle 54. The reference line 58 is a straight line having a length extending from the mitral annulus wall on the right side to the mitral annulus wall on the left side.

The condition setting unit 34 sets, as an opening line 60, a straight line obtained by translating the reference line 58 toward the left ventricle 54 by a pre-specified distance. The condition setting unit 34 sets a blood flow opening 61 on the opening line 60. In VFM, calculations based on flow rates at the blood flow opening 61 are carried out.

As such, the reference line 58 is set at the position of the mitral valve 56 so as to mark off the boundary between the left atrium 52 and the left ventricle 54. Further, the opening line 60 is set parallel to the reference line 58 at a location shifted therefrom by a pre-specified distance toward the left ventricle 54, and the blood flow opening 61 is set on the opening line 60. With this arrangement, blood flow rates can be determined at positions where the blood flow rates are relatively large within the left ventricle 54.

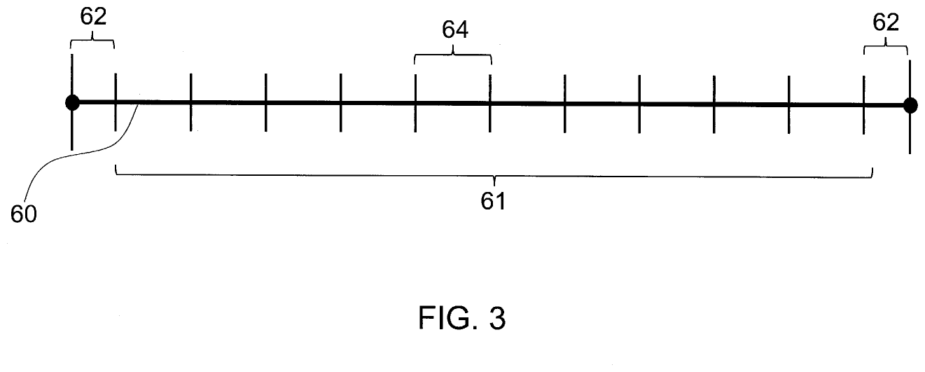

FIG. 3 conceptually illustrates a process of virtually designating flow inlets 64 with respect to the blood flow opening 61. The condition setting unit 34 sets, as the blood flow opening 61, a section of the opening line 60 that is located between the mitral annulus walls 62. Further, the condition setting unit 34 divides the blood flow opening 61 into equal parts, and each section obtained by this division is designated as a flow inlet 64. FIG. 3 shows an example in which each of 10 divided opening parts obtained by dividing the blood flow opening 61 into 10 sections is designated as the flow inlet 64. The condition setting unit 34 generates flow inlet information that indicates the direction along which the opening line 60 extends, and the ranges occupied by the respective ones of the 10 flow inlets 64.

The particle position calculator 32 executes a particle position calculation based on the flow inlet information generated by the condition setting unit 34 and the blood flow velocity data sets stored in advance in the storage device 26, and thereby determines positions and number of particles to be depicted on the tomographic image.

The particle position calculation is as described below. In the particle position calculation, among the blood flow velocity data sets sequentially obtained over an elapse of time, calculation is performed based on a preceding blood flow velocity data set and a succeeding blood flow velocity data set that are adjacent to each other in time sequence (i.e., on the time axis), to determine positions and number of particles that correspond to the succeeding blood flow velocity data set. Further, in the particle position calculation, identical processes are executed for the respective ones of the plurality of flow inlets. Here, a process executed regarding one flow inlet will be described.

The particle position calculator 32 repeatedly carries out the particle position calculation concerning the blood flow velocity data sets stored in the storage device 26. More specifically, when the storage device 26 has stored therein a first blood flow velocity data set, a second blood flow velocity data set, . . . , and an Nth blood flow velocity data set which are generated in chronological order, the particle position calculator 32 carries out the particle position calculation with respect to the first and second blood flow velocity data sets, the particle position calculation with respect to the second and third blood flow velocity data sets, the particle position calculation with respect to the third and fourth blood flow velocity data sets, . . . , and the particle position calculation with respect to the (N−1)th and Nth blood flow velocity data sets.

The particle position calculator 32 calculates a blood inflow rate at the flow inlet. The blood inflow rate is a volume of blood (determined as an area, since analysis is performed in a two-dimensional plane) that flows in via the flow inlet during a period from the point when the preceding blood flow velocity data set is generated until the point when the succeeding blood flow velocity data set is generated. The blood inflow rate [m²/one frame time interval] is calculated by multiplying the frame time interval [s] to a value obtained by multiplying the blood velocity [m/s] and the flow inlet width [m]. The frame time interval is the reciprocal of the frame rate at which the tomographic image frames and the blood flow velocity data sets are generated. The blood flow velocity is a component of the blood flow velocity vector at the flow inlet, and is a component orthogonal to the flow inlet. The blood flow velocity vector used here is the blood flow velocity vector according to the succeeding blood flow velocity data set. In the following description, the point when the preceding blood flow velocity data set is generated is referred to as the first analysis time point, and the point when the succeeding blood flow velocity data set is generated is referred to as the second analysis time point.

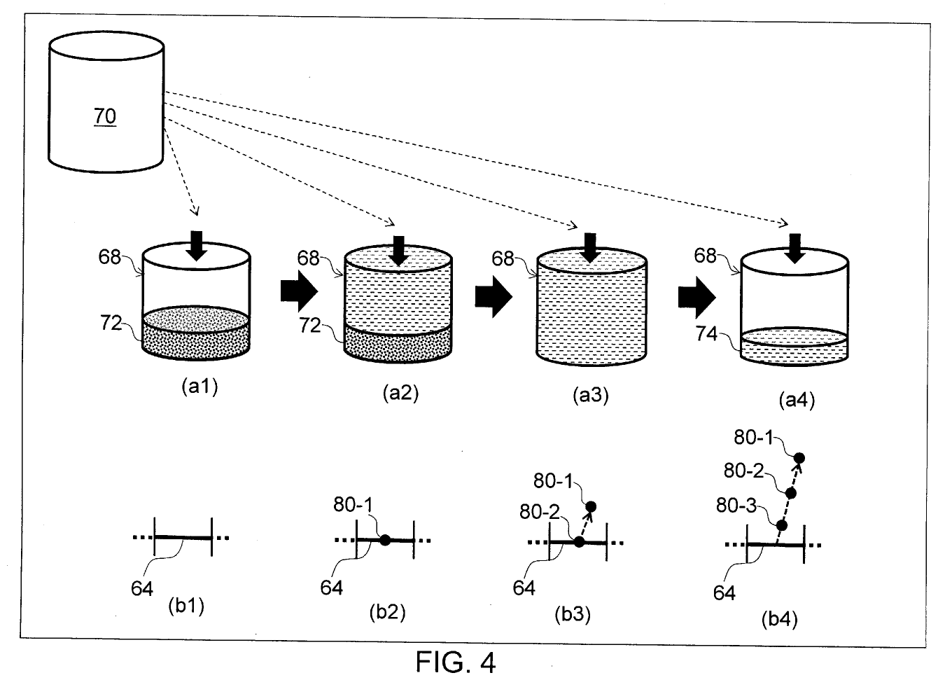

The particle position calculator 32 executes a buffer filling calculation, as described below, with respect to the blood inflow rate. FIG. 4 conceptually shows an analytical model for buffer filling calculation. The buffer filling calculation virtually employs a liquid source 70 containing an amount of liquid indicated by the blood inflow rate, and a buffer container 68 having a predetermined buffer capacity. The calculation is performed concerning the analytical model which involves performing, during a period from the first analysis time point until the second analysis time point, an operation of filling the buffer container 68 at a constant flow rate with the liquid from the liquid source 70, emptying the buffer container 68 at every instance the buffer container 68 becomes full, and continuing with the filling of the buffer container 68 at the constant flow rate with the liquid from the liquid source 70.

In the top row of FIG. 4, the liquid source 70 is shown. In the middle row of FIG. 4, states of the buffer container 68 and the liquid received in the buffer container 68 are shown in charts (a1)˜(a4) in chronological order. In the bottom row of FIG. 4, positions of particles to be depicted on the tomographic image are shown in charts (b1)˜(b4) in chronological order.

At the first analysis time point; i.e., at the point of starting one buffer filling calculation, the buffer container 68 contains an initial amount of liquid 72, as shown in chart (a1). The initial amount is identical to an amount (remaining amount) of liquid that remained in the buffer container 68 from a previously-executed buffer filling calculation. The liquid source 70 contains the amount of liquid indicated by the blood inflow rate. As shown in chart (b1), no particle is arranged on the tomographic image.

In the analytical model, the buffer container 68 is filled at a constant flow rate with the liquid received in the liquid source 70. When the buffer container 68 becomes full with the liquid as shown in chart (a2), a particle is placed at the center of the flow inlet 64 on the tomographic image. Chart (b2) shows a particle 80-1 arranged at the center of the flow inlet 64 on the tomographic image. This particle 80-1 is to be moved along with an elapse of time in the direction indicated by the blood flow velocity vector according to the succeeding blood flow velocity data set.

Chart (a3) illustrates a state in which, after the buffer container 68 is once emptied, the buffer container 68 is filled at the constant flow rate with the liquid received in the liquid source 70, and the buffer container 68 is again full with the liquid. When the buffer container 68 again becomes full with the liquid, a particle is placed at the center of the flow inlet 64 on the tomographic image. Chart (b3) shows a particle 80-2 arranged at the center of the flow inlet 64 on the tomographic image. As shown in chart (b3), when the state of the analytical model transitions from the state shown in chart (a2) to the state shown in chart (a3), the particle 80-1 is moved in the direction indicated by the blood flow velocity vector according to the succeeding blood flow velocity data set.

In the analytical model, the operation of filling the buffer container 68 at the constant flow rate with the liquid from the liquid source 70, emptying the buffer container 68 at every instance the buffer container 68 becomes full, and continuing with the filling of the buffer container 68 at the constant flow rate with the liquid from the liquid source 70 is repeated until the liquid received in the liquid source 70 becomes exhausted. At every instance the buffer container 68 becomes full, a particle is placed at the flow inlet 64, and this particle is to be moved in the direction indicated by the blood flow velocity vector according to the succeeding blood flow velocity data set.

Chart (a4) shows the liquid (i.e., the remaining amount of liquid 74) received in the buffer container 68 at the second analysis time point after the liquid received in the liquid source 70 is exhausted; i.e., at the point of completing one buffer filling calculation. This remaining amount of liquid is used as the initial value in the subsequently-executed buffer filling calculation. Chart (b4) shows the particles 80-1-80-3 that are moved from the center of the flow inlet 64 on the tomographic image.

As such, in the buffer filling calculation, a particle is generated at the flow inlet 64 every time the buffer container 68 becomes full, and position of each particle at the second analysis time point is determined, which is a position at which the particle appears when the particle is moved along an inflow direction indicated by a blood flow velocity vector until the second analysis time point is reached. The above-noted buffer capacity is a value that, in cases where a plurality of particles are to be displayed on the tomographic image, defines the distance between the particles based on the blood inflow rate.

In the buffer filling calculation, the operation of filling the buffer container 68 with the liquid after the first analysis time point is virtually carried out in a state in which the initial amount of liquid is received in the buffer container 68 at the first analysis time point. In the buffer filling calculation, the amount of liquid remaining in the liquid source 70 at the second analysis time point serves as the initial amount in a buffer filling calculation for a subsequently-executed particle position calculation.

After the particle position calculation for the preceding blood flow velocity data set and the succeeding blood flow velocity data set is executed, the display processor 38 uses the tomographic image frame corresponding to the succeeding blood flow velocity data set to thereby cause the display unit 20 to display an ultrasound image in which the respective particles are depicted on the tomographic image. In other words, the display processor 38 generates ultrasound image data for an image in which the respective particles are depicted on the tomographic image indicated according to the tomographic image frame, and causes the display unit 20 to display an ultrasound image according to the generated ultrasound image data.

When the storage device 26 has stored therein a first blood flow velocity data set and a first tomographic image frame, the second blood flow velocity data set and a second tomographic image frame, . . . , and an Nth blood flow velocity data set and an Nth tomographic image frame, which are generated in chronological order, the display processor 38 sequentially generates ultrasound image data and causes the display unit 20 to display ultrasound images according to the ultrasound image data, as described below. Here, N is an integer greater than or equal to 1.

Specifically, the display processor 38 first generates ultrasound image data based on the first blood flow velocity data set and the first tomographic image frame, and also based on the second blood flow velocity data set and the second tomographic image frame. Next, the display processor 38 generates ultrasound image data based on the second blood flow velocity data set and the second tomographic image frame, and also based on the third blood flow velocity data set and the third tomographic image frame. The display processor 38 proceeds likewise, and finally generates ultrasound image data based on the (N−1)th blood flow velocity data set and the (N−1)th tomographic image frame, and also based on the Nth blood flow velocity data set and the Nth tomographic image frame. The display processor 38 causes the display unit 20 to sequentially display images based on the respective ultrasound image data.

In the above, it has been described that the direction along which each particle is to be moved is the direction indicated by the blood flow velocity vector according to the succeeding blood flow velocity data set. The direction along which each particle is to be moved may alternatively be the direction indicated by the blood flow velocity vector according to the preceding blood flow velocity data set.

Actual computations carried out for the above-described buffer filling calculation are as described below. The position, at the second analysis time point, of the particle generated first at the center of the flow inlet 64 is determined as a position moved from the center of the flow inlet 64 by a vector obtained by multiplying the moving time move_time(1) during which that particle is moved, by the velocity vector. The moving time move_time(1) of the first particle is determined as a value obtained by multiplying the ratio of a remaining inflow amount flow_rem(1) at the time of particle generation to the blood inflow rate flow_rate, by the frame time interval flm_intvl. Here, the remaining inflow amount flow_rem(1) is the volume of liquid that remains in the liquid source 70 when the buffer container 68 is filled with the liquid and becomes full for the first time.

The volume received in the liquid source 70 at the first analysis time point is indicated by the blood inflow rate flow_rate. Assuming that the buffer capacity is denoted by flow_th and the initial amount at the first analysis time point is denoted by flow_buf, the remaining inflow amount flow_rem(1) is calculated by Formula 1.

flow_rem(1)=flow_rate−(flow_th−flow_buf) (Formula 1)

The moving time move_time(1) of the first particle is calculated by Formula 2.

move_time(1)=[flow_rem(1)/flow_rate]×flm_intvl (Formula 2)

The position, at the second analysis time point, of the particle generated first at the center of the flow inlet 64 is the position (x(1), y(1)) of the first particle at the point when the liquid source 70 becomes empty. This position (x(1), y(1)) is calculated by Formula 3. Here, (xnk, ynk) is the position coordinates of the center of the flow inlet 64, and (vxnk, vynk) is the blood flow velocity vector at the center of the flow inlet 64. This blood flow velocity vector is based on the succeeding blood flow velocity data set.

x(1)=xnk+vxnk·move_time(1)

y(1)=ynk+vynk·move_time(1) (Formula 3)

Accordingly, for the second analysis time point, the first particle is depicted on the tomographic image at the position obtained by Formula 3.

Assuming that j denotes an integer greater than or equal to 2, the volume flow_rem(j), which is the volume received in the liquid source 70 at the point when the jth particle is generated, is calculated by Formula 4.

flow_rem(j)=flow_rem(j−1)−flow_th (Formula 4)

Here, under the condition in which flow_rem(j−1)<flow_th holds true, the jth particle is not generated.

The position, at the second analysis time point, of the particle generated for the jth time at the center of the flow inlet 64 is determined as a position moved from the center of the flow inlet 64 by a vector obtained by multiplying the moving time move_time(j) during which the particle is moved, by the velocity vector. The moving time move_time(j) of the jth particle is calculated by Formula 5.

move_time(j)=[flow_rem(j)/flow_rate]×flm_intvl (Formula 5)

The position (x(j), y(j)), at the second analysis time point, of the particle generated for the jth time at the center of the flow inlet 64 is the position (x(j), y(j)) of the jth particle at the point when the liquid source 70 becomes empty. This position is calculated by Formula 6.

x(j)=xnk+vxnk·move_time(j)

y(j)=ynk+vynk·move_time(j) (Formula 6)

Accordingly, for the second analysis time point, the jth particle is depicted on the tomographic image at the position obtained by Formula 6.

The storage device 26 has stored therein a buffer filling calculation program for carrying out computations according to the above Formulas 1 to 6. The calculation device 18 virtually configures the particle position calculator 32 by executing the buffer filling calculation program, and determines the positions of the respective particles on the tomographic image.

The storage device 26 has stored therein an ultrasound image generation program including the buffer filling calculation program as described above. The calculation device 18 causes the display unit 20 to display the ultrasound image by executing the ultrasound image generation program. This program causes the calculation device 18 to carry out the following processes (i) and (ii):

(i) The process of obtaining the blood flow velocity vector at the flow inlet designated in the analysis target region, and the process of calculating, based on the blood flow velocity vector, the blood inflow rate indicating an amount of blood that flows in via the flow inlet during a period from the first analysis time point until the second analysis time point; and

(ii) The process of performing the buffer filling calculation based on the blood inflow rate and the blood flow velocity vector and thereby generating the ultrasound image for the second analysis time point. Here, the buffer filling calculation is the calculation of determining positions of particles to be shown in the ultrasound image based on the blood inflow rate, the pre-specified buffer capacity, and the blood flow velocity vector, and generating the ultrasound image in which the particles are shown at the determined positions of the particles. The buffer capacity is the value which, when a plurality of particles are to be shown in the ultrasound image, defines a distance between the particles based on the blood inflow rate.

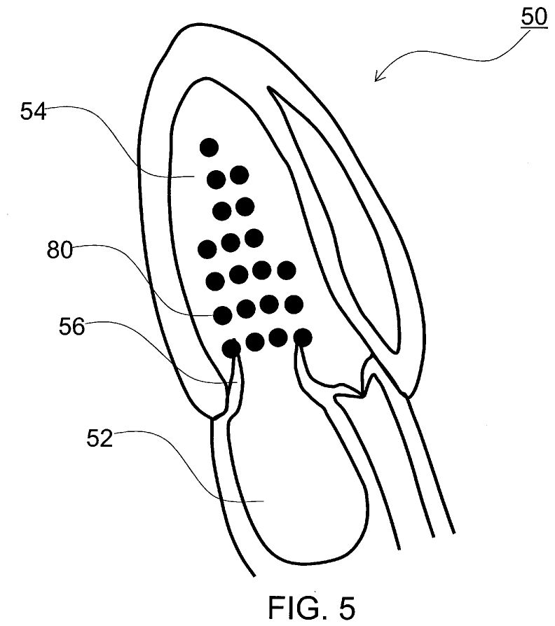

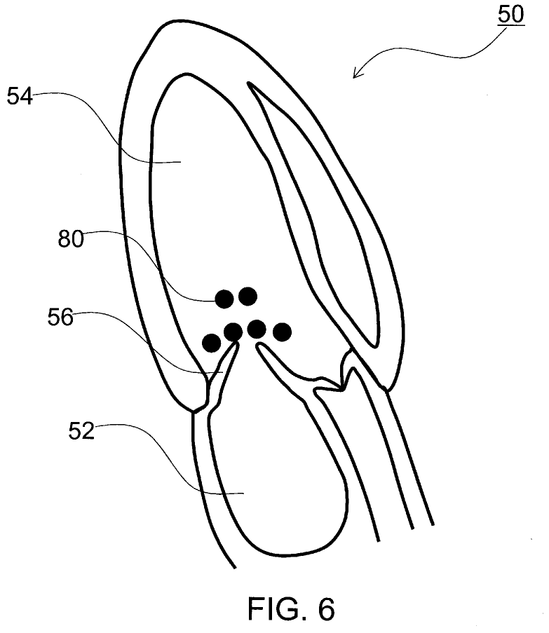

Each of FIGS. 5 and 6 schematically shows an ultrasound image in which the particles 80 indicating the rate at which blood is flowing in at that point are depicted on a tomographic image. The ultrasound image shown in FIG. 5 illustrates a heart 50 at the initial stage of diastole of the left ventricle 54. The ultrasound image shown in FIG. 6 illustrates the heart 50 at a point immediately before the last stage of diastole of the left ventricle 54.

In FIGS. 5 and 6, the rate of blood flow traveling from the mitral valve 56 to the left ventricle 54 is indicated by the plurality of particles 80 arranged in the direction from the mitral valve 56 to the left ventricle 54. In these drawings, an increased number of particles 80 arranged in the direction from the mitral valve 56 to the left ventricle 54 signifies a higher blood flow rate. These drawings show that the blood flow rate is higher toward the tip of the mitral valve 56, while the blood flow rate is lower toward the valve annulus part. Further, it is shown that, as compared to the rate of blood flow flowing in at the initial stage of diastole of the left ventricle 54, the rate of blood flow flowing in at a point immediately before the last stage of diastole becomes lower.

As described above, the ultrasonic diagnosis apparatus according to an embodiment of the present disclosure provides, in a format that facilitates user recognition, an appropriate indication of a blood flow rate at a flow inlet which is a region having an extent of area. Further, a blood flow opening set by a user is divided into a plurality of flow inlets, and a blood flow rate is calculated for each of the flow inlets. With this arrangement, distribution of blood flow rates in the analysis target region is appropriately indicated in a format that facilitates user recognition. Still further, in buffer filling calculations carried out for respective ones of repeatedly-executed particle position calculations, the remaining amount obtained from a preceding buffer filling calculation serves as the initial amount for the succeeding buffer filling calculation. With this arrangement, continuity in the sequentially-generated ultrasound images is enhanced, and the user can easily recognize the blood flow rates at the flow inlets.

The particle position calculator 32 may determine a total number (total particle number) of particles that are generated at the blood flow opening 61 during a period from the point when the first tomographic image frame is generated until the point when the Jth tomographic image frame to be displayed is generated. Here, J is an integer greater than or equal to 2 and less than or equal to N. The display processor 38 may cause the display unit 20 to display the total particle number together with the tomographic image. Further, the display processor 38 may also obtain a total inflow amount along with the total particle number, and may cause the display unit 20 to display the total inflow amount together with the tomographic image. The total inflow amount is a volume of blood that flowed in via the blood flow opening 61 during the period from the point when the first tomographic image frame is generated until the point when the Jth tomographic image frame to be displayed is generated.

Moreover, the particle position calculator 32 may determine a number (inter-frame particle number) of particles that are generated at the blood flow opening 61 during a period from the point when the (J−1)th tomographic image frame is generated until the point when the Jth tomographic image frame is generated. The display processor 38 may cause the display unit 20 to display the inter-frame particle number together with the tomographic image. Further, the display processor 38 may also obtain an inter-frame inflow amount along with the inter-frame particle number, and may cause the display unit 20 to display the inter-frame inflow amount together with the tomographic image. The inter-frame inflow amount is a volume of blood that flowed in via the blood flow opening 61 during the period from the point when the (J−1)th tomographic image frame is generated until the point when the Jth tomographic image frame is generated.

In the above, an embodiment was described in which VFM based on the particle position calculation is carried out with respect to the blood flow velocity data sets and the tomographic image frames that are sequentially generated at the frame time interval. In cases where an interpolation process is performed with respect to the blood flow velocity data sets and the tomographic image frames that are sequentially generated at the frame time interval, and interpolation data sets and interpolation frames are thereby generated for the blood flow velocity data sets and the tomographic image frames, VFM may be carried out also for the interpolation data sets and the interpolation frames. In other words, VFM based on the particle position calculation may be carried out with respect to a sequence of blood flow velocity data sets having interpolation data sets inserted therein along the time axis, and with respect to tomographic image frames having interpolation frames inserted therein along the time axis.

1. An ultrasound image generation apparatus, comprising
a processor that executes:
a process of obtaining a blood flow velocity vector at a flow inlet designated in an analysis target region;
a process of calculating, based on the blood flow velocity vector, a blood inflow rate indicating an amount of blood that flows in via the flow inlet during a period from a first analysis time point until a second analysis time point; and
a process of performing a buffer filling calculation based on the blood inflow rate and the blood flow velocity vector, and thereby generating an ultrasound image for the second analysis time point, wherein
the buffer filling calculation is a calculation of determining a number and positions of particles to be shown in the ultrasound image based on the blood inflow rate, a pre-specified buffer capacity, and the blood flow velocity vector, and generating the ultrasound image in which the particles are shown at those positions of the particles, and
the buffer capacity is a value which, when a plurality of particles are to be shown in the ultrasound image, defines a distance between the particles based on the blood inflow rate.

2. The ultrasound image generation apparatus according to claim 1, wherein
the flow inlet is one of a plurality of divided opening sections obtained by dividing a blood flow opening set in the analysis target region.

3. The ultrasound image generation apparatus according to claim 1, wherein
the buffer filling calculation is a calculation in which, when an operation of filling a buffer container, which has the buffer capacity, at a constant flow rate with a liquid from a liquid source containing the liquid by the amount indicated by the blood inflow rate, emptying the buffer container at every instance the buffer container becomes full, and continuing with the filling of the buffer container at the constant flow rate with the liquid is virtually carried out during a period from the first analysis time point until the second analysis time point, a particle is generated at the flow inlet every time the buffer container becomes full, and position of each particle at the second analysis time point is determined, which is a position at which the particle appears when the particle is moved along a direction indicated by the blood flow velocity vector until the second analysis time point is reached.

4. The ultrasound image generation apparatus according to claim 3, wherein
the processor repeatedly performs the buffer filling calculation,
in each of the buffer filling calculations, an operation of filling the buffer container with the liquid after the first analysis time point is virtually carried out in a state in which an initial amount of liquid is received in the buffer container at the first analysis time point, and
each of the buffer filling calculations is a calculation in which an amount of the liquid remaining in the liquid source at the second analysis time point serves as the initial amount for a subsequently-executed buffer filling calculation.

5. An ultrasonic diagnosis apparatus, comprising:
the ultrasound image generation apparatus according to claim 1; and
a blood flow velocity calculation device that calculates the blood flow velocity vector based on ultrasound transmissions/receptions, wherein
the processor obtains the blood flow velocity vector from the blood flow velocity calculation device.

6. The ultrasonic diagnosis apparatus according to claim 5, comprising:
a tomographic image generation device that generates a tomographic image based on ultrasound transmissions/receptions; and
a display unit that displays the ultrasound image, wherein
the ultrasound image is an image in which the particles are depicted on the tomographic image.

7. An ultrasound image generation program configured to cause a processor to execute:
a process of obtaining a blood flow velocity vector at a flow inlet designated in an analysis target region;
a process of calculating, based on the blood flow velocity vector, a blood inflow rate indicating an amount of blood that flows in via the flow inlet during a period from a first analysis time point until a second analysis time point; and
a process of performing a buffer filling calculation based on the blood inflow rate and the blood flow velocity vector, and thereby generating an ultrasound image for the second analysis time point, wherein
the buffer filling calculation is a calculation of determining a number and positions of particles to be shown in the ultrasound image based on the blood inflow rate, a pre-specified buffer capacity, and the blood flow velocity vector, and generating the ultrasound image in which the particles are shown at those positions of the particles, and
the buffer capacity is a value which, when a plurality of particles are to be shown in the ultrasound image, defines a distance between the particles based on the blood inflow rate.
